(19) United States
(12) Patent Application Publication
(10) Pub. No.: US 2025/0204769 A1
(43) Pub. Date: Jun. 26, 2025

(54) Eye Characteristic Determination

(71) Applicant: APPLE INC., Cupertino, CA (US)

(72) Inventors: Paul X. Wang, Cupertino, CA (US); Jie Gu, Palo Alto, CA (US)

(21) Appl. No.: 18/851,435

(22) PCT Filed: May 3, 2023
(86) PCT No.: PCT/US2023/020773

§ 371 Date: Sep. 26, 2024

Related U.S. Application Data
(60) Provisional application No. 63/343,132, filed on May 18, 2022.

(51) Int. Cl. A61B 3/00; A61B 3/107; A61B 3/12; A61B 3/14; G06V 10/82; G06V 40/19

(52) U.S. Cl. CPC A61B 3/005; A61B 3/107; A61B 3/12; A61B 3/14; G06V 10/82; G06V 40/19

(57) ABSTRACT

A method includes capturing a first image of an eye of a user by a first sensor coupled with a head-mounted device worn by the user and capturing a second image of the eye by a second sensor coupled with the head-mounted device. The method includes determining an eye characteristic based on the first image and the second image, and outputting a notification of the eye characteristic using an output component of the head-mounted device.

### CROSS-REFERENCE TO RELATED APPLICATIONS

This application is a National Stage Application of International Application No. PCT/US2023/020773 filed on May 3, 2023, which claims the benefit of U.S. Provisional Application No. 63/343,132, filed on May 18, 2022, the contents of which are hereby incorporated by reference in its entirety for all purposes.

### FIELD

The present disclosure relates generally to the field of head-mounted devices.

### BACKGROUND

Head-mounted devices are worn on a head of a user and may be used to show computer-generated content to the user. These devices generally include various sensors directed toward and away from a face of the user.

### SUMMARY

One aspect of the disclosure is a method that includes capturing a first image of an eye of a user by a first sensor coupled with a head-mounted device worn by the user and capturing a second image of the eye by a second sensor coupled with the head-mounted device. The method includes determining an eye characteristic based on the first image and the second image, and outputting a notification of the eye characteristic using an output component of the head-mounted device.

Another aspect of the disclosure is a method that includes capturing a first image of an eye of a user by a sensor coupled with a head-mounted device, the first image being captured at a first time. A second image of the eye is captured at a second time after the first time. The method includes determining, by a computing device, an eye characteristic by comparing the first image and the second image, and providing, by the computing device, a notification based on the eye characteristic.

Yet another aspect of the disclosure is a method that includes capturing data related to an eye of a user by an inward-facing sensor coupled with a head-mounted device. The method also includes determining, using a machine learning model that is trained to recognize indications of an eye condition, the eye of the user exhibits the eye condition based on the data, and providing a notification that the eye of the user exhibits the eye condition, the notification including a portion of the data.

### BRIEF DESCRIPTION OF THE DRAWINGS

FIG. 1 is a front view of a head-mounted device worn on a head of a user.

FIG. 2 is a cross-sectional view of the head mounted device taken along line A-A in FIG. 1

FIG. 3 is a schematic view of electronics of the head-mounted device.

FIG. 4 is a schematic view of the controller of the head-mounted device.

FIG. 5 is a flowchart of a method for providing a notification of an eye characteristic.

FIG. 6 is a flowchart of another method for providing a notification of an eye characteristic.

### DETAILED DESCRIPTION

The disclosure herein relates to a head-mounted device configured to sense eye characteristics. Sensing eye characteristic can be accomplished by using one or more sensors to capture one or more images of an eye. The images can be evaluated and compared over time to determine whether the eye characteristics are indicative of an associated eye condition. Notification of the eye characteristics and/or the associated eye condition can be provided by the head-mounted device.

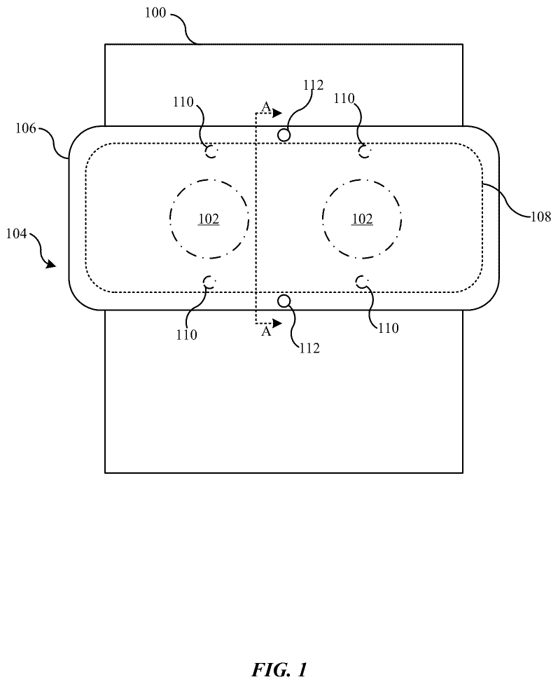

FIG. 1 is a front view of a head-mounted device 104 worn on a head of a user 100. The head-mounted device 104 is shown to include a housing 106 and a facial interface 108. The housing 106 is coupled with the facial interface 108 and functions to enclose components within the head-mounted device 104 that provide graphical content to the user 100. The housing 106 may further function to block ambient light from reaching the eyes 102 of the user 100. In addition to including the various structures that allow the user 100 to see, the eyes 102 may also include the eyelids, eye lashes, tear ducts, and any other surrounding structures. The head-mounted device 104 may also include a head support (not shown) that engages the head of the user 100 to support the housing 106. The head support may, for example, be a band that extends around the sides and rear of the head of the user 100. The head support may also include a band that extends over the top of the head of the user 100.

The facial interface 108 is coupled to the housing 106 and engages the face of the user 100 to support the head-mounted device 104. For example, the facial interface 108 may be coupled to an end of the housing 106 proximate the user 100 (e.g., a rear surface or an inward end or surface), while the head support may be in tension around the head of the user 100, thereby pressing the facial interface 108 generally rearward against the face of the user 100. The facial interface 108 may be arranged generally between the face of the user 100 and the components positioned within the head-mounted device 104.

The head-mounted device 104 is further shown to include inward-facing sensors 110 and outward-facing sensors 112. The inward-facing sensors 110 are positioned within the housing 106 and/or the facial interface 108 and are configured to sense various conditions related internal to the housing 106 and or the facial interface 108. For example, the inward-facing sensors 110 may sense conditions related to the operation of the head-mounted device 104. The inward-facing sensors 110 may also sense conditions related to the user 100 (e.g., conditions related to the health of the user 100 such as heart rate, perspiration, and/or conditions related to the appearance and/or function of the eyes 102). The inward-facing sensors 110 are further described with reference to FIG. 2. The outward-facing sensors 112 are positioned on the housing 106 and are configured to sense conditions external to the housing 106 of the head-mounted device 104. For example, the outward-facing sensors 112 may sense conditions related to the environment around the user 100. The outward-facing sensors 112 may also sense conditions related to another user 100 that is not wearing the head-mounted device 104. The outward-facing sensors 112 are further described with reference to FIG. 2.

FIG. 2 is a cross-sectional view of the head-mounted device 104 taken along line A-A in FIG. 1. As shown in FIG. 2, the head-mounted device 104 further includes a lens assembly 216 coupled to an intermediate wall 218, a display 220, and electronics 222. The intermediate wall 218 is shown to extend laterally across the head-mounted device 104. The intermediate wall 218 can be coupled to the facial interface 108 as shown in FIG. 2. The intermediate wall 218 can also be coupled to the housing 106 in other example embodiments. The lens assembly 216 includes various components that support the function of displaying content to the eyes 102 of the user 100. For example, the lens assembly 216 can include a lens that directs light from the display 220 to the eyes 102 of the user 100. In addition, the lens assembly 216 may include various adjustment assemblies that allow the lens assembly 216 to be adjusted. For example, the lens assembly 216 may be supported by an interpupillary distance adjustment mechanism that allows the lens assembly 216 to slide laterally inward or outward (e.g., toward, or away from a nose of the user 100). As another example, the lens assembly 216 may be supported by a distance adjustment mechanism that allows adjustment of the distance between the lens assembly 216 and the eyes 102. Such an adjustment mechanism may be implemented to provide eye relief and/or facilitate capturing images of the eyes 102. Furthermore, the intermediate wall 218 may also include various adjustment mechanisms to move the lens assembly 216 toward or away from the eyes 102 to facilitate capturing images of the eyes 102.

The display 220 is an output component of the head-mounted device 104 and is located between the lens assembly 216 and the electronics 222. Positioned as described, the display 220 is configured to project light (e.g., in the form of images) along an optical axis such that light is incident on the lens assembly 216 and is shaped by the lens assembly 216 such that the light projected by the display 220 is directed to the eyes 102.

The electronics 222 are electronic components for operation of the head-mounted device 104. The electronics 222 may be coupled to the display 220, for example, and are contained within the housing 106. In some embodiments, the electronics 222 may be positioned in the housing 106 but separate from the display 220. Some of the electronics 222 may be positioned remotely from the display 220 (e.g., outside of the housing 106), such as another computing device in communication with the display 220 and/or the facial interface 108.

FIG. 2 is further shown to include an inward-facing sensor 110a, an inward-facing sensor 110b, an inward-facing sensor 110c, and an inward-facing sensor 110d (collectively referred to herein as inward-facing sensors 110a-110d). Each of the inward-facing sensors 110a-110d can include any of the types of sensors described above related to the inward-facing sensors 110. In an example embodiment, the inward-facing sensors 110a-110d are cameras (e.g., visible light cameras, infrared cameras, three-dimensional cameras that can perform a three-dimensional scan, depth sensing cameras, etc.).

The inward-facing sensor 110a is shown to be coupled with the facial interface 108. Though two of the inward-facing sensor 110a are shown, more or fewer of the inward-facing sensor 110a may be implemented. The inward-facing sensor 110a may be directed to the eyes 102 such that the inward-facing sensor 110a can capture one or more images of the eyes 102. For example, multiple of the inward-facing sensor 110a may be distributed around the facial interface 108 such that each of the inward-facing sensor 110a views the eyes 102 of the user 100 from a different angle. Each of the images acquired by each inward-facing sensor 110a can be combined to render a comprehensive image of the eyes 102. The inward-facing sensor 110a may also be directed to other portions of the face of the head-mounted device 104 such that the inward-facing sensor 110a can capture one or more images of the face of the user 100.

The inward-facing sensor 110b is shown to be coupled with the intermediate wall 218. Though two of the inward-facing sensor 110b are shown, more or fewer of the inward-facing sensor 110b may be implemented. The inward-facing sensor 110b may be directed to the eyes 102 such that the inward-facing sensor 110b can capture one or more images of the eyes 102. For example, multiple of the inward-facing sensor 110b may be distributed around the facial interface 108 such that each of the inward-facing sensor 110b views the eyes 102 of the user 100 from a different angle. Each of the images acquired by each inward-facing sensor 110b can be combined to render a comprehensive image of the eyes 102. The inward-facing sensor 110b may also be directed to other portions of the face of the head-mounted device 104 such that the inward-facing sensor 110b can capture one or more images of the face of the user 100. In some implementations, the position of the inward-facing sensors 110b may be adjusted toward or away from the eyes 102 by adjusting the position of the intermediate wall 218.

The inward-facing sensor 110c is shown to be coupled to a front surface of the display 220 (e.g., a surface of the display 220 that is closest to the user 100). The inward-facing sensor 110c is positioned on the display 220 such that the inward-facing sensor 110c can capture an image of the eyes 102 through the lens assembly 216. Accordingly, by moving the lens assembly 216 relative to the eyes 102, such as toward or away from the eyes 102 (e.g., via the adjustment mechanisms described), and/or by adjusting a focal length of the inward-facing sensor 110c, the inward-facing sensor 110c may be able to focus on different areas of the eyes 102 when capturing an image of the eyes 102. For example, the inward-facing sensor 110c can capture images of a lens, a retina, an optic nerve, a macula, and/or a vitreous body of the eyes 102. Though one inward-facing sensor 110c is shown, more than one inward-facing sensor 110c may be implemented. For example, multiple of the inward-facing sensor 110c may be distributed around the display 220 such that the eyes 102 may be viewed from various angles to generate a comprehensive image of the eyes 102.

The inward-facing sensor 110d is shown to be coupled to a rear surface of the display 220 (e.g., a surface of the display that is furthest from the user 100). In some embodiments, the inward-facing sensor 110d is positioned adjacent to an opening in the display 220 such that the inward-facing sensor 110d can sense the eyes 102 of the user 100. In some embodiments, the display 220 is configured to make one or more pixels of the display 220 transparent such that the inward-facing sensor 110d can sense the eyes 102 of the user 100 through the transparent pixel(s). Though one inward-facing sensor 110d is shown, more than one inward-facing sensor 110d may be implemented. For example, multiple of the inward-facing sensor 110d may be distributed around the rear of the display 220 such that the eyes 102 may be viewed from various angles (e.g., through various transparent pixels on the display 220) to generate a comprehensive image of the eyes 102.

In various embodiments, the inward-facing sensors 110a-110d sense eye characteristics related to the eyes 102 of the user 100. The eye characteristics can be related to eye conditions such as eye strain, overuse, and/or fatigue. For example, the inward-facing sensors 110a-110d are configured to sense eye characteristics related to eye strain, overuse, and/or fatigue such as blink rate, finger wiping, squinting, eye open rate, and eye lash condition.

Combinations of one or more of the inward-facing sensors 110a-110d can be used. For example, the head-mounted device 104 may include at least one of the inward-facing sensor 110a, at least one of the inward-facing sensor 110b, at least one of the inward-facing sensor 110c, and at least one of the inward-facing sensor 110d. The head-mounted device 104 can also include the inward-facing sensors 110a-110d in only three of the four positions described, only two of the of the four positions described, and only one of the four positions described.

The images captured by the inward-facing sensors 110a-110d may also be used to control content creation and display. For example, the inward-facing sensors 110-110d are also configured to track movement of the eyes 102 and a focal point of the eyes 102 such that the head-mounted device 104 can determine an interaction between the user 100 and the environment surrounding the user 100 and display content on the display 220 according to the determined interaction.

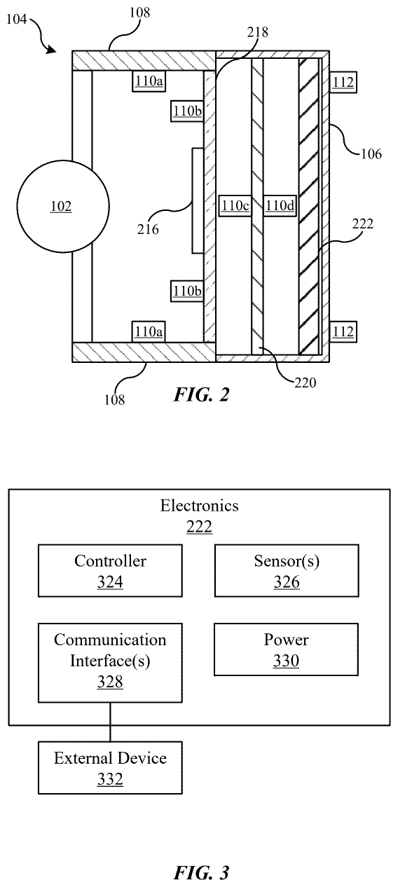

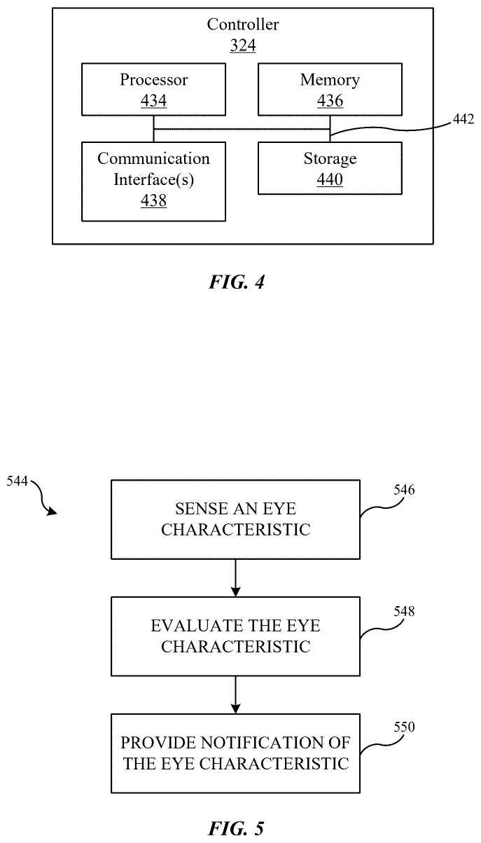

FIG. 3 is a schematic view of the electronics 222 of the head-mounted device 104. The electronics may generally include a controller 324, sensors 326 (e.g., the inward-facing sensors 110a-110d and/or the outward-facing sensors 112), a communication interface 328, and power electronics 330, among others. The electronics 222 may also be considered to include the display 220. The controller 324 generally controls operations of the head-mounted device 104, for example, receiving input signals from the sensors 326 and/or the communication interface 328 and sending control signals to the display 220 for outputting the graphical content. An example hardware configuration for the controller 324 is discussed below with reference to FIG. 4. The sensors 326 sense conditions of the user 100 (e.g., physiological conditions), the head-mounted device 104 (e.g., position, orientation, movement), and/or the environment (e.g., sound, light, images). The sensors 326 may be any suitable type of sensor like the ones described with reference to the inward-facing sensors 110a-110d and the outward-facing sensors 112. The communication interface 328 is configured to receive signals from an external device 332 that is physically separate from the head-mounted device 104. The power electronics 330 store and/or supply electric power for operating the head-mounted device 104 and may, for example, include one or more batteries. The external device 332 may be a user input device (e.g., a user controller), another electronic device associated with the user 100 (e.g., a smartphone or a wearable electronic device), or another electronic device not associated with the user 100 (e.g., a server, smartphone associated with another person). The external device 332 may include additional sensors that may sense various other conditions of the user 100, such as location or movement thereof. The external device 332 may be considered part of a display system that includes the head-mounted device 104.

FIG. 4 is a schematic view of the controller 324 of the head-mounted device 104. The controller 324 may be used to implement the apparatuses, systems, and methods disclosed herein. For example, the controller 324 may receive various signals from various electronic components (e.g., the sensors 326 and the communications interface 328) and control output of the display 220 according thereto to display the graphical content. In an example hardware configuration, the controller 324 generally includes a processor 434, a memory 436, a storage 440, a communication interface 438, and a bus 442 by which the other components of the controller 324 are in communication. The processor 434 may be any suitable processor, such as a central processing unit, for executing computer instructions and performing operations described thereby. The memory 436 may be a volatile memory, such as random-access memory (RAM). The storage 440 may be a non-volatile storage device, such as a hard disk drive (HDD) or a solid-state drive (SSD). The storage 440 may form a computer readable medium that stores instructions (e.g., code) executed by the processor 434 for operating the head-mounted device 104, for example, in the manners described above and below. The communications interface 438 is in communication with other electronic components (e.g., the sensors 326, the communications interface 328, and/or the display 220) for sending thereto and receiving therefrom various signals (e.g., control signals and/or sensor signals).

FIG. 5 is a flowchart of a method 544 for providing a notification of an eye characteristic. The method 544 may be implemented by, for example, the controller 324 and/or the electronics 222. At operation 546, an eye characteristic is sensed. For example, the user 100 may be wearing the head-mounted device 104. Upon activating the head-mounted device 104 (e.g., turning on the power to the head-mounted device 104), the inward-facing sensors 110a-110d and the outward-facing sensors 112 are powered on and are active. The inward-facing sensors 110a-110d are positioned as described above and are configured to sense an eye characteristic. The eye characteristic sensed may be any one or more of the eye characteristics described above. In some embodiments, the inward-facing sensors 110a-110d sense the eye characteristic by capturing one or more images of the eyes 102 of the user 100. The inward-facing sensors 110a-110d may store the one or more images in an internal memory. The inward-facing sensors 110a-110d may also store data related to the one or more images in the internal memory. In some embodiments, the inward-facing sensors 110a-110d do not store images or data related thereto and provide the images and/or the data related to the images to the controller 324. In some embodiments, the controller 324 stores the images and/or the data related thereto in the memory 436.

At operation 548, the eye characteristic is evaluated. For example, the controller 324 receives the images from the inward-facing sensors 110a-110d and evaluates the eye characteristic based on analysis of the images. In some embodiments, the images are evaluated using a trained machine learning model that has been trained to analyze images of an eye and determine an eye characteristic associated with the eye. The trained machine learning model may include a trained neural network. Training the machine learning model may be accomplished by providing images of various eye characteristics to the controller 324, where the images of the various eye characteristics are tagged with the specific eye characteristic represented by the images. The machine learning model can then be tested by challenging the model to categorize additional images of eyes that are untagged. Upon categorizing the image, the machine learning model is notified whether the determined category is correct or incorrect, and the machine learning model updates internal image evaluation algorithms accordingly. Using this method, the machine learning model learns how to accurately categorize eye characteristics based on images received from the inward-facing sensors 110a-110d and the outward-facing sensors 112. Thus, determining the eye characteristic may be performed using the trained neural network, which receives images as an input.

The eye characteristic is evaluated by the controller 324 comparing an image received from the inward-facing sensors 110a-110d to another image received from the inward-facing sensors 110a-110d (e.g., comparing multiple images received from various inward-facing sensors 110a-110d) and/or to images of known eye characteristics. For example, the images received may indicate that the eyes 102 of the user 100 have characteristics such as bulging, excessive blinking, and discharge. After comparing the received images to each other and comparing the received images to stored images and/or known characteristics, the controller 324 determines the eyes 102 exhibit one or more eye characteristics based on the comparison. In some implementations, the determination is made based on a similarity between the images. The determination can also be made based on a difference between the images (e.g., a difference between the images received from the inward-facing sensors 110a-110d). In some embodiments, the controller 324 may determine that the one or more eye characteristics correspond to one or more eye conditions and that the eyes 102 exhibit (e.g., show) the one or more eye conditions. Using the above example, the controller 324 may determine that the bulging, excessive blinking, and discharge may be associated with eye conditions such as keratoconus and conjunctivitis.

At operation 550, notification of the eye characteristic is provided. For example, the controller 324 may operate the display 220 to notify the user 100 of the eye characteristic. In some implementations, the display 220 may notify the user 100 with text superimposed over the graphics being displayed to the user 100. The display 220 may also replace the graphics being displayed to the user 100 with text providing the notification of the eye characteristic. In some embodiments, the display 220 may include both text and images, where the text provides notification of the eye characteristic and at least a portion of an image of the eye that indicates the condition. For example, the display 220 may provide the user 100 a notification of eye bulging and may also provide an image of the eyes 102 of the user 100 that shows the bulging. The display 220 may also display an image of an eye that does not exhibit bulging along with an image of the eyes 102 of the user 100 that does exhibit bulging. This type of notification can be used in conjunction with any of the eye characteristics described herein to notify the user 100 of the eye characteristic. In some implementations, notification of the eye characteristic may include a notification of both the eye characteristic and the potential eye condition(s) that are associated with the eye characteristic. Using the above example, the display 220 may provide the user 100 a notification of eye bulging and provide an image of the eyes 102 and may concurrently provide the user 100 a notification that the bulging eyes may indicate that the user 100 has an eye condition like keratoconus.

In some embodiments, the controller 324 may determine that an additional image of the eye may be needed to evaluate the eye characteristic or an additional eye characteristic. In such cases, the controller 324 may control the display 220 to prompt the user 100 to capture an additional image of the eyes 102 with an additional sensor (e.g., the outward-facing sensors 112). To do so, the user 100 may need to remove the head-mounted device 104 and turn the head-mounted device 104 around such that the outward-facing sensors 112 face the eyes 102 of the user 100 and can capture the additional image. Upon successfully capturing the additional image, the user 100 may be notified by an audio or visual notification that the image has been successfully captured so the user 100 can put the head-mounted device 104 back on. The controller 324 may then evaluate and determine the eye characteristic and/or the additional eye characteristic using the methods described above. The display 220 may then output an additional notification of the additional eye characteristic. For example, the additional eye characteristic may be associated with redness of the eyes 102 of the user 100, and the notification may include text and an image of the eyes 102 of the user 100 showing the redness level of the eyes 102 along with a notification that redness of the eyes 102 may be associated with an eye condition like a sty.

In some embodiments, the notification may include information not only regarding the determined eye characteristic, but also a prompt for the user 100 to take an action based on the determined eye characteristic. For example, the eye characteristic may indicate that the user 100 has an eye condition like the eye conditions described above. In such cases, the prompt may include a message directing the user 100 to contact a clinician to professionally evaluate the eye characteristic and potential eye condition related to the eye characteristic.

In some implementations, the action may be related to eye fatigue. For example, the eye characteristic may indicate the user 100 is not blinking enough or wiping the eyes 102 excessively, indicating the eyes 102 may be fatigued. Eye fatigue can result from focusing on the same plane for an extended duration without changing the focal plane. Accordingly, the controller 324 may direct the display 220 to provide an instruction that directs the user 100 to blink the eyes 102 and/or follow a virtual object displayed on the display 220 with the eyes 102. The display 220 may move the virtual object to simulate three-dimensional movement of the object that causes the eyes 102 of the user 100 to focus on different virtual planes to reduce eye fatigue.

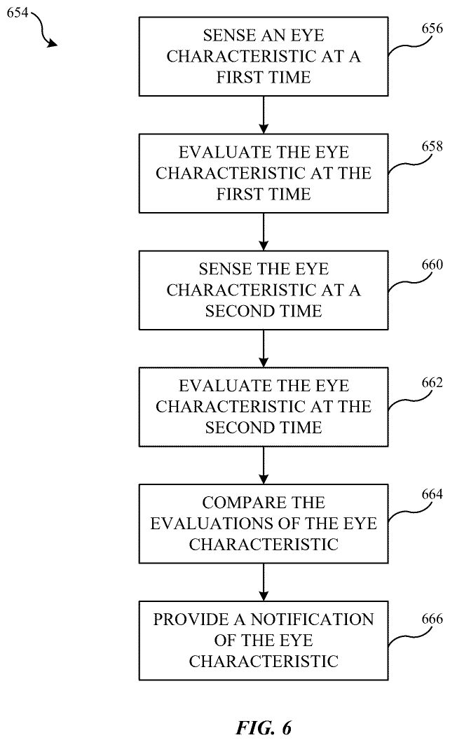

FIG. 6 is a flowchart of another method 654 for providing a notification of an eye characteristic. The method 654 may be implemented by, for example, the controller 324 and/or the electronics 222. At operation 656, an eye characteristic is sensed at a first time, and at operation 658, the eye characteristic is evaluated at the first time. The sensing and evaluation of the eye characteristic at operations 656 and 658 are similar to operations 546 and 548 of FIG. 5, and the descriptions of operations 546 and 548 also apply to operations 656 and 658. After sensing and evaluating the eye characteristic at the first time, the controller 324 may store images of the eyes 102 and data related to the images of the eyes 102 in the memory 436 to be retrieved later. The data related to the images of the eyes 102 may include data indicative of the eye characteristic and/or data indicative of one or more eye conditions associated with the eye characteristic.

At operation 660, the eye characteristic is sensed at a second time. For example, the user 100 may wear the head-mounted device 104 at a second time after the first time. The duration between the first time and the second time can be any duration during which the eye characteristic may change. In some implementations, the duration between the first time and the second time can be on the order of minutes (e.g., five minutes, ten minutes, fifteen minutes, etc.). In some implementations, the duration between the first time and the second time can be on the order of hours (e.g., one hour, two hours, five hours, ten hours, etc.). The duration between the first time and the second time can also be on the order of days (e.g., one day, two days, three days, etc.). In some embodiments, the duration between the first time and the second time can be on the order of weeks (e.g., one week, two weeks, three weeks, etc.). In some embodiments, the duration between the first time and the second time can also be on the order of months (e.g., one month, two months, three months, etc.). The duration between the first time and the second time can also be on the order of years (e.g., one year, two years, three years, etc.). The eye characteristic is sensed in the same manner as described above with respect to operation 546.

At operation 662, the eye characteristic is evaluated at the second time. The eye characteristic is evaluated in the same manner as described above with respect to operation 548.

At operation 664, the evaluations of the eye characteristic at the first time and the second time are compared. For example, the eye characteristic may include an image of the retina captured via a three-dimensional scan. The controller 324 may retrieve from the memory 436 one or more images of the retina (and/or data related to the images) captured at the first time and compare the one or more images captured at the first time with one or more images (and/or data related to the images) of the retina captured at the second time. The comparison may include comparing the properties of the retina (e.g., shape, size, color, blood vessel distribution, etc.) of the images captured at the first time with those of the images captured at the second time.

In some instances, the comparison may indicate that the eye characteristic has not changed from the first time to the second time. If the eye characteristic is indicative of an eye condition, the user 100 may have exhibited the eye condition at the first time and continues to exhibit eye condition at the second time. If the eye characteristic is not indicative of an eye condition, the user 100 may not have an eye condition.

In some embodiments, the comparison may indicate that the eye characteristic has changed from the first time to the second time. The eye characteristic may not have been indicative of an eye condition at the first time, but the eye condition may be indicative of an eye condition at the second time, indicating that the user 100 has developed the eye condition between the first time and the second time. For example, an image of the corneas of the eyes 102 at the first time may show the corneas to be clear with no sign of a cataract, and an image of the corneas of the eyes 102 at the second time may show the corneas to be cloudy, which indicates the eyes 102 may have cataracts.

In some implementations, the images are evaluated and/or compared using a trained machine learning model that has been trained to analyze images of an eye, compare images of the eye captured over time, and determine an eye characteristic associated with the eye. The trained machine learning model may include a trained neural network that is trained in the same manner described with reference to operation 548. Thus, determining the eye characteristic may performed using a trained neural network that receives a first image and a second image as inputs.

At operation 666, notification of the eye characteristic based on the comparison is provided. For example, the controller 324 may operate the display 220 to notify the user 100 of the eye characteristic. In some implementations, the display 220 may notify the user 100 with text superimposed over the graphics being displayed to the user 100. The display 220 may also replace the graphics being displayed to the user 100 with text providing the notification of the eye characteristic. In some embodiments, the display 220 may include both text and images, where the text provides notification of the eye characteristic and at least a portion of the images captured at the first time and the second time that shows the eye condition. For example, the display 220 may provide the user 100 a notification that the user 100 may have cloudy corneas and may also provide an image of the eyes 102 of the user 100 at the first time (which shows the corneas being clear) and at the second time (which shows the corneas being cloudy). This type of notification can be used in conjunction with any of the eye characteristics described herein to notify the user 100 of the eye characteristic. In some implementations, the notification of the eye characteristic may include a notification of both the eye characteristic and the potential eye condition(s) that are associated with the eye characteristic. Using the above example, the display 220 may provide the user 100 a notification of cornea cloudiness and provide an image of the eyes 102 and may concurrently provide the user 100 a notification that the cloudy corneas may indicate that the user 100 has an eye condition like glaucoma.

In addition to providing the notification, the controller 324 may also direct the display 220 to prompt the user 100 to take an action based on the notification. The prompt may include an instruction for the user 100 to contact a clinician for a professional evaluation of the eye condition. The controller 324 may also provide contact information for clinicians located near the user 100 and may communicate with the external device 332 (e.g., a mobile device of the user 100) to automatically call a clinician chosen by the user 100 or to automatically schedule an appointment with the clinician chosen by the user 100.

The prompt may also include prompts for the user 100 to take other actions based on the comparison. For example, if the comparison shows that the eyes 102 of the user 100 have become drier or redder over time, the prompt may include an instruction for the user 100 to use eye drops to reduce the redness and increase lubrication. If the comparison shows that the eyes 102 of the user 100 have become fatigued over time, the prompt may include an instruction for the user 100 to remove the head-mounted device 104 to rest the eyes 102 or for the user 100 to follow a simulated object on the display 220 as the simulated object moves virtually in three dimensions on the display 220.

The system and methods described above may also be applied to sensing, evaluating, and determining an eye characteristic of an additional user that is not wearing the head-mounted device 104. For example, the user 100 may notify the additional user that the head-mounted device 104 can evaluate eye characteristics. Upon receiving consent of the additional user, the user 100 may direct the controller 324 to direct the outward-facing sensors 112 to capture images of the eyes of the additional user. The images of the eyes of the additional user can be evaluated in the same manner described above. Notification of the evaluation and determination of the eye characteristic and any associated eye condition and/or actions that should be taken based on the determination can be sent to the user 100 via the display 220. In some embodiments, the user 100 may indicate that the notification should be provided to the mobile device of the additional user and provide the contact information for the additional user to the head-mounted device 104. Providing the notification to the additional user via the mobile device of the additional user can avoid providing the notification to the user 100 to maintain privacy.

A physical environment refers to a physical world that people can sense and/or interact with without aid of electronic systems. Physical environments, such as a physical park, include physical articles, such as physical trees, physical buildings, and physical people. People can directly sense and/or interact with the physical environment, such as through sight, touch, hearing, taste, and smell.

In contrast, a computer-generated reality (CGR) environment refers to a wholly or partially simulated environment that people sense and/or interact with via an electronic system. In CGR, a subset of a person's physical motions, or representations thereof, are tracked, and, in response, one or more characteristics of one or more virtual objects simulated in the CGR environment are adjusted in a manner that comports with at least one law of physics. For example, a CGR system may detect a person's head turning and, in response, adjust graphical content and an acoustic field presented to the person in a manner similar to how such views and sounds would change in a physical environment. In some situations (e.g., for accessibility reasons), adjustments to characteristic(s) of virtual object(s) in a CGR environment may be made in response to representations of physical motions (e.g., vocal commands).

A person may sense and/or interact with a CGR object using any one of their senses, including sight, sound, touch, taste, and smell. For example, a person may sense and/or interact with audio objects that create three-dimensional or spatial audio environment that provides the perception of point audio sources in three-dimensional space. In another example, audio objects may enable audio transparency, which selectively incorporates ambient sounds from the physical environment with or without computer-generated audio. In some CGR environments, a person may sense and/or interact only with audio objects.

Examples of CGR include virtual reality and mixed reality.

A virtual reality (VR) environment refers to a simulated environment that is designed to be based entirely on computer-generated sensory inputs for one or more senses. A VR environment comprises a plurality of virtual objects with which a person may sense and/or interact. For example, computer-generated imagery of trees, buildings, and avatars representing people are examples of virtual objects. A person may sense and/or interact with virtual objects in the VR environment through a simulation of the person's presence within the computer-generated environment, and/or through a simulation of a subset of the person's physical movements within the computer-generated environment.

In contrast to a VR environment, which is designed to be based entirely on computer-generated sensory inputs, a mixed reality (MR) environment refers to a simulated environment that is designed to incorporate sensory inputs from the physical environment, or a representation thereof, in addition to including computer-generated sensory inputs (e.g., virtual objects). On a virtuality continuum, a mixed reality environment is anywhere between, but not including, a wholly physical environment at one end and virtual reality environment at the other end.

In some MR environments, computer-generated sensory inputs may respond to changes in sensory inputs from the physical environment. Also, some electronic systems for presenting an MR environment may track location and/or orientation with respect to the physical environment to enable virtual objects to interact with real objects (that is, physical articles from the physical environment or representations thereof). For example, a system may account for movements so that a virtual tree appears stationery with respect to the physical ground.

Examples of mixed realities include augmented reality and augmented virtuality.

An augmented reality (AR) environment refers to a simulated environment in which one or more virtual objects are superimposed over a physical environment, or a representation thereof. For example, an electronic system for presenting an AR environment may have a transparent or translucent display through which a person may directly view the physical environment. The system may be configured to present virtual objects on the transparent or translucent display, so that a person, using the system, perceives the virtual objects superimposed over the physical environment. Alternatively, a system may have an opaque display and one or more imaging sensors that capture images or video of the physical environment, which are representations of the physical environment. The system composites the images or video with virtual objects, and presents the composition on the opaque display. A person, using the system, indirectly views the physical environment by way of the images or video of the physical environment, and perceives the virtual objects superimposed over the physical environment. As used herein, a video of the physical environment shown on an opaque display is called “pass-through video,” meaning a system uses one or more image sensor(s) to capture images of the physical environment, and uses those images in presenting the AR environment on the opaque display. Further alternatively, a system may have a projection system that projects virtual objects into the physical environment, for example, as a hologram or on a physical surface, so that a person, using the system, perceives the virtual objects superimposed over the physical environment.

An augmented reality environment also refers to a simulated environment in which a representation of a physical environment is transformed by computer-generated sensory information. For example, in providing pass-through video, a system may transform one or more sensor images to impose a select perspective (e.g., viewpoint) different than the perspective captured by the imaging sensors. As another example, a representation of a physical environment may be transformed by graphically modifying (e.g., enlarging) portions thereof, such that the modified portion may be representative but not photorealistic versions of the originally captured images. As a further example, a representation of a physical environment may be transformed by graphically eliminating or obfuscating portions thereof.

An augmented virtuality (AV) environment refers to a simulated environment in which a virtual or computer-generated environment incorporates one or more sensory inputs from the physical environment. The sensory inputs may be representations of one or more characteristics of the physical environment. For example, an AV park may have virtual trees and virtual buildings, but people with faces photorealistically reproduced from images taken of physical people. As another example, a virtual object may adopt a shape or color of a physical article imaged by one or more imaging sensors. As a further example, a virtual object may adopt shadows consistent with the position of the sun in the physical environment.

There are many different types of electronic systems that enable a person to sense and/or interact with various CGR environments. Examples include head-mounted systems, projection-based systems, heads-up displays (HUDs), vehicle windshields having integrated display capability, windows having integrated display capability, displays formed as lenses designed to be placed on a person's eyes (e.g., similar to contact lenses), headphones/earphones, speaker arrays, input systems (e.g., wearable or handheld controllers with or without haptic feedback), smartphones, tablets, and desktop/laptop computers. A head-mounted system may have one or more speaker(s) and an integrated opaque display. Alternatively, a head-mounted system may be configured to accept an external opaque display (e.g., a smartphone). The head-mounted system may incorporate one or more imaging sensors to capture images or video of the physical environment, and/or one or more microphones to capture audio of the physical environment. Rather than an opaque display, a head-mounted system may have a transparent or translucent display. The transparent or translucent display may have a medium through which light representative of images is directed to a person's eyes. The display may utilize digital light projection, OLEDs, LEDs, uLEDs, liquid crystal on silicon, laser scanning light source, or any combination of these technologies. The medium may be an optical waveguide, a hologram medium, an optical combiner, an optical reflector, or any combination thereof. In one embodiment, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface.

As described above, one aspect of the present technology is the gathering and use of data available from various sources for use during operation of the head-mounted device 104. As an example, such data may identify the user 100 and include user-specific settings or preferences. The present disclosure contemplates that in some instances, this gathered data may include personal information data that uniquely identifies or can be used to contact or locate a specific person. Such personal information data can include demographic data, location-based data, telephone numbers, email addresses, twitter ID's, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, a user profile may be established that stores medical related information that allows comparison of eye characteristics. Accordingly, use of such personal information data enhances the user's experience.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of storing a user profile for comparison of eye characteristics over time, the present technology can be configured to allow users to select to “opt in” or “opt out” of participation in the collection of personal information data during registration for services or anytime thereafter. In another example, users can select not to provide data regarding usage of specific applications. In yet another example, users can select to limit the length of time that application usage data is maintained or entirely prohibit the development of an application usage profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, an eye characteristic may be determined each time the head-mounted device 104 is used, such as by capturing images of the eyes 102 with the inward-facing sensors 110 and/or the outward-facing sensors 112, and without subsequently storing the information or associating with the particular user.

1. A method, comprising:
capturing a first image of an eye of a user by a first sensor coupled with a head-mounted device worn by the user;
capturing a second image of the eye by a second sensor coupled with the head-mounted device;
determining an eye characteristic based on the first image and the second image; and
outputting a notification of the eye characteristic using an output component of the head-mounted device.

2. The method of claim 1, wherein the first sensor is an inward-facing sensor and the second sensor is an outward-facing sensor.

3. The method of claim 2, wherein after capturing the first image, an additional notification is provided using the output component, the additional notification including a prompt to capture the second image with the second sensor.

4. The method of claim 1, wherein the notification includes providing a prompt to the user to take an action based on the eye characteristic.

5. The method of claim 4, wherein the prompt to take the action includes an instruction directing the user to focus on an object shown on the output component, wherein the output component moves the object to simulate three-dimensional movement of the object.

6. The method of claim 1, wherein capturing the first image includes performing a three-dimensional scan of a shape of the eye.

7. The method of claim 1, wherein determining the eye characteristic is performed using a trained neural network that receives the first image as an input.

8. The method of claim 1, wherein the eye characteristic indicates eye fatigue.

9. The method of claim 1, wherein capturing the first image includes:
positioning the first sensor on the head-mounted device such that a lens of the head-mounted device is positioned between the eye and the first sensor;
moving the lens relative to the eye to allow the first sensor to sense a retina of the eye; and
capturing the first image, wherein the first image is an image of the retina.

10. The method of claim 1, wherein the output component is a display.

11. The method of claim 1, wherein determining the eye characteristic includes:
determining a difference between the first image and the second image; and
determining the eye characteristic based on the difference.

12. A method, comprising:
capturing a first image of an eye of a user by a sensor coupled with a head-mounted device, the first image being captured at a first time;
capturing a second image of the eye, the second image being captured at a second time after the first time;
determining, by a computing device, an eye characteristic by comparing the first image and the second image; and
providing, by the computing device, a notification based on the eye characteristic.

13. The method of claim 12, wherein determining the eye characteristic is performed using a trained neural network that receives the first image and the second image as inputs.

14. The method of claim 12, wherein the notification includes an instruction directing the user to focus the eye on an object shown on a display of the head-mounted device.

15. The method of claim 14, wherein the display simulates three-dimensional movement of the object.

16. The method of claim 12, wherein the notification includes an instruction directing the user to blink the eye.

17. A method, comprising:
capturing data related to an eye of a user by an inward-facing sensor coupled with a head-mounted device;
determining, using a machine learning model that is trained to recognize indications of an eye condition, the eye of the user exhibits the eye condition based on the data; and
providing a notification that the eye of the user exhibits the eye condition, the notification including a portion of the data.

18. The method of claim 17, wherein the eye condition includes eye strain.

19. The method of claim 17, wherein the eye condition includes eye fatigue.

20. The method of claim 19, wherein providing the notification includes providing an instruction directing the user to focus on an object shown on a display of the head-mounted device, wherein the display simulates three-dimensional movement of the object.
